(19) United States
(12) Patent Application Publication
(10) Pub. No.: US 2023/0218327 A1
(43) Pub. Date: Jul. 13, 2023

(54) ELASTICALLY DEFORMABLE FASTENING DEVICE

(71) Applicants: UNIVERSITE DE BORDEAUX, BORDEAUX (FR); INSTITUT POLYTECHNIQUE DE BORDEAUX, TALENCE CEDEX (FR); CENTRE NATIONAL DE LA RECHERCHE SCIENTIFIQUE, PARIS CEDEX 16 (FR); ECOLE NATIONALE SUPERIEURE DES ARTS ET METIERS, PARIS (FR); UNIVERSITY OF AVEIRO, AVEIRO (PT)

(72) Inventors: MICHEL MESNARD, BORDEAUX (FR); ANTONIO RAMOS, ILHAVO (PT)

(21) Appl. No.: 18/003,508

(22) PCT Filed: Jun. 28, 2021
(86) PCT No.: PCT/EP2021/067742

§ 371 Date: Dec. 28, 2022

(30) Foreign Application Priority Data
Jun. 29, 2020 (FR) FR2006780

(51) Int. Cl. A61B 17/84; A61C 8/00

(52) U.S. Cl. CPC A61B 17/844; A61C 8/0033

(57) ABSTRACT

The invention relates to a fastening device for an implant that is elastically deformable by stress, the length of the device in the unstressed state being less than that of the device in the stressed state, and the diameter of the device in the unstressed state being greater than that of the device in the stressed state, and to a kit comprising it.

### TECHNICAL FIELD

The present invention lies in the field of medical devices, and relates more particularly to an elastically deformable fastening device intended to be inserted into a housing, or a cavity, in particular formed in a bone in order to allow the fitting of an implant. The invention also relates to a kit comprising such a fastening device, to the associated implant and to an elastic deformation means for allowing the fitting of said device in said housing.

### TECHNOLOGICAL BACKGROUND

Loss of bone may occur as a result of simple ageing of the tissue, as a result of bone traumas, or even as a result of curative surgical procedures.

In certain surgical procedures, the devices are generally fastened in the bone tissue by screwing, or are implanted in previously drilled holes and are expanded using mechanical means in order to be locked in the bone. It has also been demonstrated that, by treating the surface of the material used, such as titanium, by blasting, chrome pickling, electrical discharge machining, laser bombardment, etc., so as to create micro-rugosities, the adhesion of bone cells to the implant is promoted, accelerating the formation of bone on the implant and therefore healing, even in the case of a low-density bone.

However, the dynamics of bone healing are variable data depending on the individual and on the anatomical, biological (age, medications, etc.), pathological (bacterial, viral), behavioural (smoking, hygiene, addictions, etc.), traumatic (fitting of an implant) circumstances, and it is difficult to predict the success of the implantation.

The causes of failure are difficult to dissociate from the quality of the bone tissue but the improvement of osseointegration cannot be researched simply by modifying the states and treatments of the surfaces of the implant.

The lines of improvement for the implanted devices are linked to the reduction/elimination of the loss of bone around the implant, and to the thoroughness/quality of execution of the protocol by the practitioner.

Thus, in spite of the efforts undertaken to date, there remains a need for systems and methods that are advantageous and effective and provide improved bone implantation and stabilization assemblies.

Therefore, the present invention aims to overcome the abovementioned drawbacks and to propose a novel implant fastening device which can be fastened robustly and durably in the bone.

### SUMMARY OF THE INVENTION

The invention relates to a device intended to be used as an anchoring system for allowing the fastening of an implant in or on bones of the skeleton of a, human or animal, mammal, and the methods for fitting such a device. The invention relates more specifically to a fastening device that is able to change shape during its use, and more particularly at the time that it is fitted.

The invention deals with a fastening device intended to be interposed between a bone tissue and an implant (functional element) so as to promote the osseointegration of said implant and to reduce the loss of bone around the implant. This fastening device makes it possible to improve the connection to the bone tissue in order to promote a durable and robust implantation of said device. More specifically, the fastening device according to the invention, as a result of its elastic deformations, allows micro-movements of the device with respect to the surrounding tissue, thereby promoting the reconstruction of the bone tissue around said device and reducing the risks of the loss of bone around the implant. This fastening device is notably particularly suitable for the osseointegration of dental implants.

The device according to the invention has a tubular overall shape, forming a housing delimited by a wall made of flexible material. The part of the device comprising said housing, which is elastic and deformable, is intended to be housed and retained in a bone cavity and to receive the actual implant. The geometry of the device, and more particular of the part of the device comprising the housing, is adapted to the bone cavity in which it is intended to be housed. Thus, for example, in dentistry, the external contours of the part of the device comprising the housing will closely follow the contours of the tissue bordering the cavity made in the bone tissue next to the tooth to be replaced by a dental implant. The geometry (thickness, height, structure) of the fastening device advantageously takes account of the surrounding bone tissue, of the loads and deformations to which it will be subjected, etc.

The objective of the invention is to simplify bone recovery by limiting the difficulties of the surgical procedures and by reducing the associated costs.

By allowing an elastic connection with the surrounding bone tissue, the fastening device according to the invention makes it possible:
to make the whole implantation process simpler and more reliable, and/or
to integrate the mechanical characteristics of the materials and of the structure, and/or
to ensure load transfer, taking into account the characteristics of the bone tissue, and/or
to manage the intra- and inter-variability of the mechanical characteristics of the bone tissue, and/or
to ensure the fastening and primary stabilization and the osseointegration and secondary bone/implant connection, thereby allowing the durable and robust implantation of the device, and/or
to eliminate the loss of bone around the implant and thus to improve the seal with respect to bacteria.

The invention also relates to kits, methods and tools that are usable for the fitting of the fastening devices and implants.

The invention relates more particularly to a fastening device for implants, comprising an open proximal part, a distal part and a central hollow body extending between the proximal part and the distal part, said hollow body forming a housing extending along a longitudinal axis X, said device being made of elastic material and said hollow body being elastically deformable by axial elongation along the longitudinal axis X so as to pass from an unstressed state into a stressed state and vice versa, wherein a length L of the hollow body in the unstressed state is less than a length L′ of said hollow body in the stressed state, and a diameter D of the hollow body in the unstressed state is greater than a diameter D′ of said hollow body in the stressed state.

In the context of the invention, “distal” and “proximal” are understood in relation to the position intended to be adopted by the fastening device once it has been fitted in a bone cavity. Thus, the distal end of the device is the end furthest away from the mouth of the bone cavity, the proximal part being the part intended to be flush with or to extend out of said cavity when the device is in position in the cavity.

The terms “internal” and “external” are understood in relation to the housing formed in the hollow body of the device. An element referred to as internal is therefore situated inside said housing, or more generally inside the device, with an external element being situated outside said housing, or more generally outside the device.

The term “implant” is understood to be any single structure of structural assembly configured to be connected or fastened to the fastening device. Typically, a dental implant is understood to be the structure carrying the false tooth, which is inserted into the fastening device.

The term “prosthesis” is understood to be the assembly formed by at least one implant, at least one fastening device and optionally the other elements that make it up, the aim of which is to be placed in the body in order to restore a compromised function. For example, a dental prosthesis comprises a fastening device according to the invention and a dental implant, the dental implant bearing the crown (false tooth) being inserted into the fastening device. Examples of prostheses according to the invention are illustrated in FIGS. 6, 7 and 8.

The length of the hollow body denotes the greatest dimension of said hollow body, extending along the longitudinal axis X. The diameter of the hollow body denotes the greatest dimension of said hollow body extending in a plane transverse to the longitudinal axis X.

Preferably, the hollow body has a tubular overall shape.

Advantageously, the wall of the hollow body delimiting the housing is perforated. For example, the wall of the hollow body comprises at least one longitudinal slot and/or at least one helical slot. In one embodiment, the wall of the hollow body comprises at least two helical slots with identical, different or opposite pitches. In another embodiment, the wall of the hollow body is in the form of a lattice.

Advantageously, the distal part of the fastening device, which is intended to be in contact with the bottom of the bone cavity, has a conical or frustoconical cylindrical shape. In one embodiment, a distal end of the distal part is closed. In another embodiment, the distal end of the distal part is pierced, meaning that a through-orifice is provided in a wall of said end, allowing the interior of the hollow body to be in communication with the external environment (and more particularly the bone cavity) into which said fastening device will be inserted. Advantageously, the distal part of the fastening device forms a base, which is optionally pierced. In a preferred embodiment, an internal surface of the distal part is substantially smooth, meaning that it does not have any thread or other fastening surface.

According to one aspect, an internal surface of the wall of the hollow body comprises a fastening surface intended for fastening an implant in said hollow body. Advantageously, the fastening surface extends along a partial length of the wall of the hollow body. For example, the fastening surface extends along at most ¼, at most ⅓ or at most ½ of the length of the hollow body. In particular, the fastening surface extends in a proximal part of the hollow body, forming the junction with the proximal part of the fastening device. Alternatively, the fastening surface intended for fastening an implant in said hollow body is provided in the proximal part of the fastening device.

According to the invention, an “elastic material” is understood to be a material that is able to deform elastically when it is subjected to an external stress, such as tension, compression, torsion, etc.

Preferably, an elastic material is understood to be a material having a Young's modulus of between 10⁴ and 10¹¹ Pascal. Advantageously, the elastic material of which all or part of the device according to the invention is made is one of a polymer, titanium or one of its alloys, preferably an alloy of titanium, niobium and zirconium. Of course, since the device is intended to be used in an animal body, the material is chosen from biocompatible materials, or biomaterials. A person skilled in the art is capable of choosing a suitable elastic material, depending in particular on the stresses to which the device will be subjected as a result of its use.

When the elastic material is titanium or one of its alloys, the device according to the invention advantageously exhibits a variation in length between the length L and the length L′ of between 0.1 and 2 millimetres and/or a variation in diameter between the diameter D and the diameter D′ of between 0.01 and 1 millimetre.

According to one aspect, when the elastic material is titanium or one of its alloys, the wall of the hollow body may typically have a yield strength in tension of between 10⁸ and 10¹⁰ Pascal, and a Young's modulus of between 2×10¹⁰ and 30×10¹⁰ Pascal, in particular a Young's modulus of between 4×10¹⁰ and 30×10¹⁰ Pascal, preferably between 6.5×10¹⁰ and 7×10¹⁰ Pascal.

In one embodiment, the housing of the hollow body is able to receive a dental implant. In other embodiments, the housing of the hollow body is able to receive an orthopaedic implant.

The invention also relates to a kit comprising a fastening device according to the invention and an elastic deformation means, such as a screw or a piston, configured to elastically deform the hollow body of the fastening device by axial elongation such that said hollow body passes from an unstressed state to a stressed state or vice versa.

The invention will be understood better from reading the following description and from studying the figures that accompany it. These are presented by way of entirely non-limiting indication of the invention.

### DESCRIPTION OF THE DRAWINGS

FIG. 1 is a schematic depiction of one embodiment, in a side view (A), in longitudinal section along the axis X (B) or in a ¾ view from the front and the right (C), of the fastening device according to the invention, according to which the fastening device has a frustoconical shape and has longitudinal slots.

FIG. 2 shows the steps of implanting the fastening device in a bone cavity, according to one of the embodiments of the invention.

FIG. 3 is a schematic depiction of a fastening device according to the invention of frustoconical shape having helical slots in a side view (A), a view in longitudinal section along the axis X (B) or a ¾ view from the front and the right (C).

FIG. 4 is a schematic depiction of embodiments of a fastening device according to the invention, according to which the wall of the fastening device has a lattice.

FIG. 5 is schematic depiction in longitudinal section along the axis X of two embodiments of the distal part of the fastening device according to the invention, having a closed distal end (A), a pierced distal end (B).

FIG. 6 is a schematic depiction of an embodiment of the fastening device according to the invention, allowing the fitting of a dental prosthesis.

FIG. 7 is a schematic depiction of an embodiment of the fastening device according to the invention, allowing the fitting of a knee prosthesis.

FIG. 8 is a schematic depiction of an embodiment of the fastening device according to the invention, allowing the fitting of a finger joint prosthesis.

### DETAILED DESCRIPTION OF THE INVENTION

Fastening Device and Kit Comprising it

The present invention proposes a fastening device for implants, comprising an open proximal part, a distal part and a central hollow body extending between the proximal part and the distal part. The device according to the invention is advantageously integral, meaning that it is made in one piece. Alternatively, the device comprises a plurality of elements that are secured together. In particular, the proximal part and/or the distal part may be attached and fastened to the central part.

In particular, the fastening device is an endosseous device allowing the fastening of an orthopaedic implant or of a dental implant.

In particular, the implant is a bone implant, such as an orthopaedic implant, for example an articular implant, in particular an articular implant of the finger or knee, or a dental implant.

The wall of the hollow body forms a hollow enclosure, a housing or a cavity or a hole in the fastening device, which extends along a longitudinal axis X. This cavity is intended to receive an implant. Advantageously, the fastening surface intended to keep the implant in position in the cavity extends over only a part of the hollow body. For example, if the fastening surface comprises a thread for screwing an implant, said thread extends along only a partial length of the internal surface of the hollow body, in particular along ¼, ⅓ or ½ of the length of the internal surface of the hollow body.

The device according to the invention is made from an elastic material. In particular, the wall of the hollow body of the device is made of elastic material and is elastically deformable by axial elongation along a longitudinal axis X, so as to pass from an unstressed state to a stressed state and vice versa.

The term “elastic deformation” is understood here as meaning the property of a solid material to return to its original shape after it has been deformed or stressed. Elastic deformation is reversible deformation, generally proportional to the stress applied. In particular, the fastening device according to the invention deforms when stresses or forces are applied to it and returns to its initial shape and geometry when these stresses are no longer exerted. Thus, by application of tensile forces, the device according to the invention can be stretched along its longitudinal axis and returns to its initial shape at rest. This stretched or extended position corresponds to the stressed or tensioned state or position of the fastening device. The original position, the return position or the rest position corresponds to the unstressed state or position.

In this context, the length L of the hollow body represents its average length in the unstressed state. The length L′ of the hollow body represents its average length in the stressed state. This length is measured along the longitudinal axis X, which extends to either side of the proximal and distal ends of the device.

The diameter D of the hollow body corresponds to the diameter in the unstressed state. The diameter D′ corresponds to the diameter of the hollow body in the stressed state. This diameter is measured at the cross section of the hollow body, in particular at the most central part of the hollow body.

In particular, the elastic material forming the hollow body of the fastening device is such that it allows an increase in length between a length L in the unstressed state and a length L′ in the stressed state, meaning that the length L′ is greater than the length L. For the devices intended to receive a dental implant, this variation in length is advantageously between 0.1 and 2 millimetres, preferably between 0.1 and 1 millimetre.

The passage from the unstressed state to the stressed state also causes a variation in the size of the diameter of the device according to the invention, between a diameter D in the unstressed state and a diameter D′ in the stressed state. In particular, the diameter D′ is smaller than the diameter D. For the devices intended to receive a dental implant, this variation in diameter between the diameter D and the diameter D′ is advantageously between 0.01 and 1 millimetre, preferably between 0.1 and 0.5 millimetre.

Advantageously, the central part of the hollow body is the most deformable part of the fastening device. In one embodiment, the wall of the hollow body may have a smaller thickness in the most central part of the hollow body compared with the thickness of said wall on either side of this most central part, in order to make it even easier to deform. Thickness is understood to be the dimension of the wall extending perpendicularly to the longitudinal axis X.

According to one embodiment, the diameter D of the hollow body in its most central part is greater than the diameter of said hollow body on either side of this most central part. Generally, the diameter of the hollow body is advantageously greater than that of the distal end of the fastening device.

In one embodiment, the central part of the hollow body comprises a shoulder or a bulge so as to locally increase the diameter of the central part.

The fastening device has a cross section of any shape, such as a round, ovoid, square, polygonal or triangular shape, adapted to the formation of a cavity of an internal housing. Preferably, the cross section of the device has a round or ovoid shape.

According to one embodiment, the fastening device may have any overall shape adapted to the implantation zone, that is to say to the bone cavity into which it is intended to be inserted. In particular, the fastening device has a tubular, cylindrical, cylindro-conical, conical or frustoconical overall shape. Preferably, the hollow body of the device has a tubular, cylindrical, cylindro-conical, conical or frustoconical overall shape.

The distal part of the fastening device according to the invention may have conical or frustoconical cylindrical shape, in particular in order to make it easier to implant in the bone. Advantageously, the distal part of the fastening device may have a tapered distal end in order to be inserted into a bone, such as a spongy bone not prepared by a drill.

According to the invention, the distal part of the fastening device is not intended to keep an implant in position, and so the internal surface of said distal part does not have any fastening surface.

The distal end of the distal part of the fastening device may be closed, open or pierced. Advantageously, the distal end of the device is pierced, in order to allow in particular the circulation of fluids and gases through the fastening device.

According to one aspect, the proximal part of the fastening device comprises a hollow fastening head via which an implant will be inserted into the hollow body of the fastening device. Generally, the proximal part is open and opens into the housing of the hollow body. The opening in the fastening head extends advantageously in the continuation of the housing of the hollow body.

A person skilled in the art knows how to adapt the thickness and the shape of the proximal part depending on the type of implant and the implantation zone. For example, the proximal part comprises a fastening head of flared or frustoconical shape, or in the shape of a flat disc or washer. The proximal part may also have, at least locally, a diameter greater than the average diameter of the hollow body, in order to reduce the risks of damaging the surrounding tissue and of preventing the fastening device from being introduced fully into the cavity formed in the bone. In particular, the fastening head may come to bear against the bone wall which borders the mouth of the cavity formed in the bone.

According to one aspect, the fastening device is integral. In this context, the whole of the device is made of elastic material.

Alternatively, the fastening device comprises at least two blocks, one formed by the fastening head, and another comprising the hollow body and optionally the distal part (which may otherwise form a third block). In this context, the fastening head may be made of elastic or rigid material, the hollow body always being made of elastic material.

In one embodiment, all or part of the external surface of the fastening device may have a rough appearance. The rough surface state promotes the adhesion and the proliferation of cells, allowing improved osseointegration of the fastening device. According to the invention, the external surface of the fastening device does not have a thread. Specifically, the fastening device is intended to be kept in a bone cavity by close contact with the surrounding tissue, and in no case by being screwed into said bone tissue.

Advantageously, the wall of the hollow body delimiting the housing is perforated, meaning that it is pierced with one or more holes, slots or the like, through the thickness of the wall. This has in particular the aim of allowing the bone cells to penetrate into the housing and to improve the keeping of the fastening device in place in the cavity formed in the bone.

Preferably, the wall of the hollow body comprises at least one longitudinal slot. In one embodiment, the wall of the hollow body is perforated by means of a plurality of longitudinal slots. Alternatively, the wall of the hollow body comprises at least one helical slot. More particularly, the wall of the hollow body may comprise at least two helical slots with identical, different or opposite pitches.

According to one embodiment, the external surface of the wall of the hollow body has ribs, or longitudinal protuberances. These ribs make it possible to locally change the flexibility by increasing the rigidity of the hollow body at said ribs. The ribs also make it possible to adjust the contact of the device with the wall of the cavity formed in the bone and the act on the fastening device/bone tissue contact zones.

To compensate for this increase in rigidity, the ribs or protuberances may be locally perforated. Alternatively or additionally, the ribs may alternate with longitudinal slots. These ribs may be provided on all the types of fastening device envisaged here.

In one embodiment, the wall of the hollow body is in the form of a lattice, of a meshing or of a grid. The meshes of the lattice may have a round, square or triangular shape depending on the application envisaged. The pattern of the meshing may be more or less tight depending on the shape and the size of the fastening device.

The internal surface of the wall of the hollow body may comprise a fastening surface intended for fastening an implant and keeping it in position in said hollow body.

Any mechanical fastening system may be envisaged, in particular by screwing or nesting. For example, the internal surface of the wall of the hollow body may have a screw pitch, or thread, that is able to receive an implant having a complementary screw shape. Alternatively, a system of coinciding threads, clips or teeth may also be envisaged. In another embodiment, the internal surface of the wall of the hollow body has striations recessed into the thickness of the wall and intended to receive a filling material in order to keep the implant in the housing by sealing.

According to one particular embodiment, the opening in the proximal part is connected to the housing of the hollow body via a neck comprising a means for fastening an implant. In a preferred embodiment, only said neck comprises a means for fastening the implant, the rest of the hollow body not having such fastening means. More generally, the means for fastening the implant in the hollow body advantageously only extend along a partial length of said hollow body.

Any mechanical fastening system may be envisaged, in particular by screwing or nesting. For example, the internal surface of the neck situated between the opening in the proximal part and the hollow body may have a screw pitch that is able to receive an implant having a complementary screw pitch. Alternatively, a system of coinciding threads, clips or teeth may also be envisaged. In a preferred embodiment, the neck situated between the opening in the proximal part and the hollow body comprises a thread extending from said proximal part towards the hollow body.

Any biocompatible material which has structural properties and is elastically deformable may be envisaged for forming the device according to the invention, such as nickel and its alloys, nitinol, titanium and its alloys including niobium and/or zirconium, stainless steel, polymers, such as polyether ether ketone, elastomers of silicone and of polyethylene, without being limited thereto.

In particular, the elastic material forming the fastening device is one of a polymer, titanium or one of its alloys, preferably an alloy of titanium, niobium and zirconium. Very particularly, the material forming the fastening device is an alloy based on titanium, niobium and zirconium, which preferably allows a reduction in the Young's modulus (Young's modulus of around 80 GPa).

According to one particular embodiment, the elastic material of which the fastening device is made, in particular the wall of the hollow body, has a yield strength in tension of between 10⁸ and 10¹⁰ Pascal.

According to one particular embodiment, the elastic material of which the fastening device is made, in particular the wall of the hollow body, has a Young's modulus of between 2×10¹⁰ and 30×10¹⁰ Pascal.

According to one embodiment, the fastening device may be covered with a composition that promotes implantation. Such a composition advantageously coats the external surface and/or the internal surface of the device. This composition may, for example, contain one or more antibiotics, in order to decontaminate the implant site; an unsaponifiable matter and/or one or more vitamins for promoting collagen healing, one or more growth factors for accelerating healing, one or more haemostatic components, and any combination including these.

The invention also relates to a kit comprising at least one fastening device as described above and an elastic deformation means, such as a screw or a piston, configured to elastically deform the hollow body of the fastening device by axial elongation or traction such that said hollow body passes from an unstressed state to a stressed state. The kit may also comprise a means for gripping the device, such as a forceps, in order to make it easier to insert the fastening device into the bone.

According to one embodiment, the kit comprises at least one fastening device according to the invention and one or more reusable or disposable surgical instruments for drilling the bone, actuate the fastening device and place it in the bone and/or fit an implant.

The kit may also comprise at least one implant, in particular an orthopaedic implant, for example an articular implant of the knee or of a finger joint, or a dental implant. The implant may be in one piece of formed of several interconnected pieces.

In particular, the kit comprises:
a fastening device according to the invention,
optionally, an instrument intended for bone preparation, in particular an instrument for drilling the bone intended to receive the fastening device according to the invention,
optionally an implant, preferably a dental implant or an articular implant of the finger or knee,
optionally, an instrument for gripping and manipulating the fastening device and/or the implant,
optionally an elastic deformation means.

The invention also deals with a prosthesis, comprising at least one fastening device according to the invention. The prosthesis also comprises at least one implant, intended to be inserted into the fastening device.

According to one embodiment, the prosthesis comprises a fastening device according to the invention and an implant. In particular, in the field of dentistry, the prosthesis is a dental prosthesis, and comprises a fastening device according to the invention and a dental implant, such as a crown or false tooth.

According to one embodiment, the prosthesis comprises two fastening devices according to the invention. In particular, the prosthesis comprises two fastening devices according to the invention and at least one articular implant. Preferably, the prosthesis comprises two fastening devices according to the invention and two complementary implants that are able to form an articular joint.

It should be noted that the present fastening device can be used for any type of joint of a subject, in particular in order to allow the joining of two bone ends and to restore a compromised function of the body.

In particular, the prosthesis according to the invention may be a prosthesis of a finger joint, such as an interphalangeal joint or metacarpophalangeal joint. In this context, the fastening device according to the invention is particularly designed to receive an implant intended to replace the metacarpophalangeal joint. Thus, the articular prosthesis is made up of two fastening devices according to the invention and of two complementary implants, one taking the form of the distal part of a metacarpus and the other taking the form of the proximal part of the first phalanx. According to one particular embodiment, the implant intended to be inserted into the fastening devices is in one piece and comprises an artificial metacarpophalangeal joint made up of the above-described elements.

Alternatively, the prosthesis according to the invention may be a knee joint prosthesis. In this context, the fastening device according to the invention is particularly designed to receive an implant intended to replace the knee joint. Thus, the articular prosthesis is made up of two fastening devices according to the invention and of two complementary implants, one taking the form of the distal part of the femur and the other the form of the proximal part of the tibia. The prosthesis may also comprise an artificial patella. According to one particular embodiment, the implant intended to be inserted into the fastening devices is in one piece.

Methods

The invention also relates to a method for implanting the fastening device according to the invention in a bone. According to one embodiment, the invention relates to a surgical method for fitting a fastening device for an implant in a bone. The invention also relates to a surgical method for fitting an implant via the fastening device according to the invention.

In one embodiment, the method comprises a preliminary step in which an implant housing is formed in a bone.

The production of the implant housing intended to receive the fastening device according to the invention may particularly involve a drilling step during which the bone is bored.

Of course, depending on the quality of the bone tissue encountered and the configuration of the implant zone, it is conceivable either to punch the bone directly or to bore a simple circular housing by way of a drill. A person skilled in the art knows how to adapt the technique to the implant zone envisaged.

In particular, the diameter of the implant housing recessed into the bone corresponds substantially to the diameter D′ of the fastening device in the extended state, that is to say in the stressed state, so as to allow the device to be introduced into the implant housing with very limited forces and stresses on the surrounding tissue.

According to the method of the invention, the device is stretched along a longitudinal axis X, so as to increase its length and reduce its diameter. The elongation of the device is carried out using any type of compatible means known to a person skilled in the art, in particular by axial traction, preferably by means of an instrument allowing deformation, such as a screw or a piston.

The method then comprises a step of fitting the fastening device in the implant housing formed in the bone.

The practitioner can in particular introduce the fastening device with the aid of a suitable gripping instrument, for example of the forceps type.

According to the method of the invention, the device is then released in the housing and adopts a less stressed or unstressed shape. The relaxation of the device makes it possible to lock the fastening device by close contact with the wall of the bone cavity. Specifically, by returning to or approaching its initial, unstressed, diameter, the device according to the invention comes into contact with the surrounding tissue and fits in the bone cavity. The relaxation of the device causes radial compression of the surrounding tissue, ensuring stabilization and adhesion at the bone tissue/fastening device interface. This primary mechanical connection is similar to a press-fit type fastening, which is controlled.

During use, in standard service, the external wall of the device ensures load transfer and, by deformation, allows relative device/tissue micro-movements. These micro-movements promote the continuous reconstruction of the bone tissue and eliminate the risk of loss of bone around the implant.

Optionally, the method according to the invention also comprises the fastening of an implant in the fastening device, for example by screwing.

During the fitting of the implant, the fastening device may also be deformed, such that the introduction of the implant increases the diameter of the fastening device, further adjusting the connection of the fastening device to the bone in which it is implanted.

Fastening Device for a Dental Implant and Kit Comprising it

According to one embodiment, the fastening device is intended for the fitting of a dental implant. In this context, the housing of the hollow body is able to receive a dental implant.

According to one embodiment, the fastening device is intended to be housed in a cavity formed in an alveolar bone in order to receive a dental implant. The outside diameter of the hollow body may be between 2 and 6 millimetres, preferably between 2.5 and 5 millimetres, at the most central part of the hollow body. Preferably, the housing formed in the hollow body of such a device has a diameter of between 2 and 5.5 millimetres, preferably between 2 and 5 millimetres.

Additionally or alternatively, the length of the hollow body of such a device is between 5 and 14 millimetres, preferably between 6 and 12 millimetres. Preferably, the housing formed in the hollow body has a length of between 5 and 11 millimetres, preferably between 7 and 10 millimetres.

In such an embodiment, the side wall of the hollow body forming the housing has a thickness of between 0.1 and 0.6 millimetre, and preferably around 0.3 millimetre.

When the fastening device intended to receive a dental implant comprises a fastening head, said head may, for example, have a diameter of between 3 and 8 millimetres and/or a length of 1 to 5 millimetres. Generally, the diameter of the fastening head is advantageously greater than the diameter of the hollow body.

In the scope of a dental implant, the implantation protocol is highly alleviated. Hitherto, the drilling/screwing operation for a fastening device for a dental implant required highly precise execution and continuous monitoring of the heating of the tissue and of the tightening torque. With the device according to the invention, the preparation of the bone site (drilling) remains analogous to that of the prior art, but the adaptive geometry of the fastening device tolerates a defect in the geometry of the bore.

The invention also aims to provide a novel surgical kit which is very easy to use and allows quick, easy and safe employment of a fastening device according to the invention and of a dental implant.

In particular, the kit comprises:
a fastening device according to the invention,
optionally, an instrument intended for bone preparation, in particular an instrument for drilling the bone intended to receive the fastening device according to the invention,
optionally, an instrument for gripping and manipulating the fastening device,
optionally an elastic deformation means,
optionally an implant, preferably a dental implant.

When the fastening device is intended to be implanted in a jaw, the device is entirely introduced into and covered by the bone, apart from the proximal part of the device, which is flush with the level of the gum.

### Detailed Description of Different Particular Embodiments of the Fastening Device According to the Invention

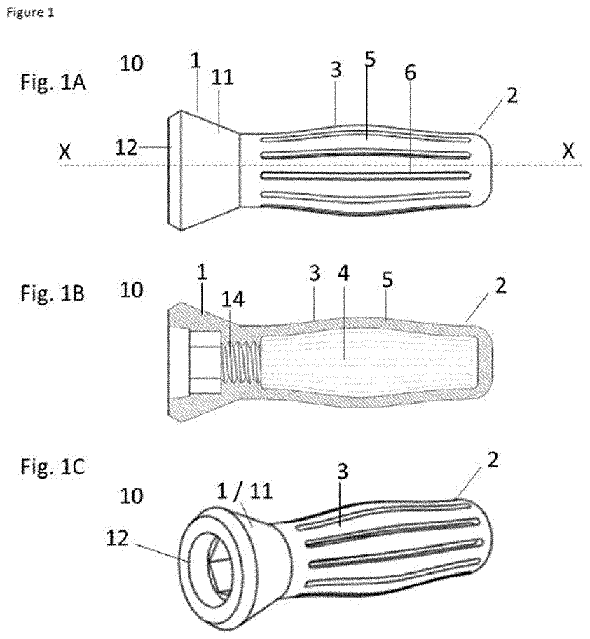

The embodiments depicted in FIGS. 1 to 5 have shared elements which are denoted by the same reference numerals. These shared elements are described in more detail below with reference to one embodiment, but apply to all of the embodiments.

FIGS. 1A, 1B and 1C depict a fastening device (10) for an implant, in particular for a dental implant, according to a first embodiment of the invention. This device (10) has a tubular overall shape and comprises an open proximal part (1), a closed distal part (2) and a central hollow body (3) extending between the proximal part (1) and the distal part (2), said hollow body (3) forming a housing (4) and extending along a longitudinal axis X.

The proximal part (1) is open such that the opening (12) in said proximal part opens into the housing (4) of the hollow body (3). Advantageously, the opening extends in the axial continuation of the housing. The distal part (2) forms a closed base.

In this embodiment, the proximal part (1) of the fastening device is formed of a flared head (11). The wall (5) of the hollow body (3) is perforated by means of a plurality of longitudinal slots (6).

According to one particular embodiment of the invention, as can be seen in FIG. 1B, the opening (12) in the proximal part is connected to the housing (4) of the hollow body (3) via a neck comprising a thread (14) extending from said proximal part towards the proximal part of the hollow body (3). Apart from the neck (14), the internal surface of the hollow body (3) is substantially smooth, meaning that it may comprise micro-rugosities but it does not have any fastening means, in particular any means for fastening an implant. Thus, the implant is kept in position in the fastening device (10) by the neck (14).

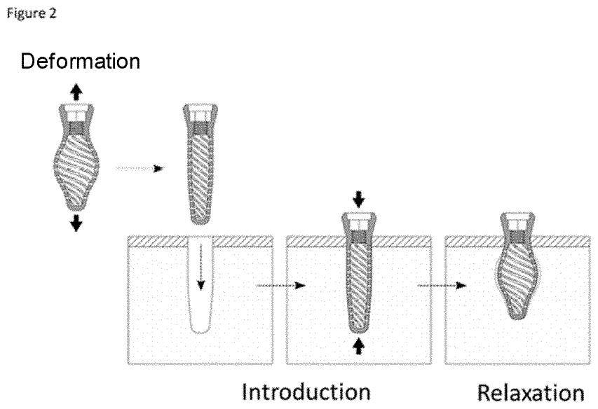

FIG. 2 presents the steps for fitting a fastening device according to the invention in a cavity, such as a bone cavity. In particular, the fastening device is deformed elastically by traction in order to reduce its diameter. The device is then introduced into the preformed cavity. Once inserted, the device relaxes and returns to a shape that tends to be identical to its initial shape (length and diameter). The relaxation of the device causes radial compression of the surrounding tissue, ensuring stabilization and adhesion at the bone tissue-fastening device interface.

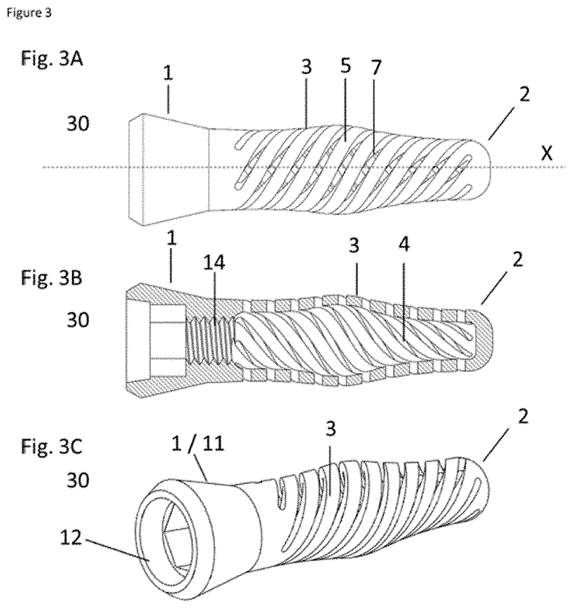

FIGS. 3A, 3B and 3C depict another embodiment of the fastening device (30) according to the invention, in particular for a dental implant, according to a second embodiment of the invention. This device (30) has a frustoconical overall shape. More specifically, in this embodiment, the hollow body (3) has a frustoconical overall shape with a reduction in diameter from the proximal part to the distal part. The hollow body (3) also has a bulge, characterized by a local increase in the diameter, at a central part of the hollow body. Thus, the diameter of the central part of the hollow body is greater than that of the rest of the hollow body on either side of this bulge.

In this embodiment, the wall (5) of the hollow body (3) is perforated by means of a plurality of helical slots (7) with the same pitch. This type of helical slot is particularly suitable for tensile-torsional deformations. Helical slots with opposite or different pitches may also be envisaged in order to adapt the device to the desired deformations.

According to one particular embodiment of the invention, as can be seen in FIG. 3B, the opening (12) in the proximal part is connected to the housing (4) of the hollow body (3) via a neck comprising a thread (14) extending from said proximal part towards the proximal part of the hollow body (3). Here too, apart from the neck (14), the internal surface of the hollow body (3) is substantially smooth, meaning that it may comprise micro-rugosities but it does not have any fastening means, in particular any means for fastening an implant.

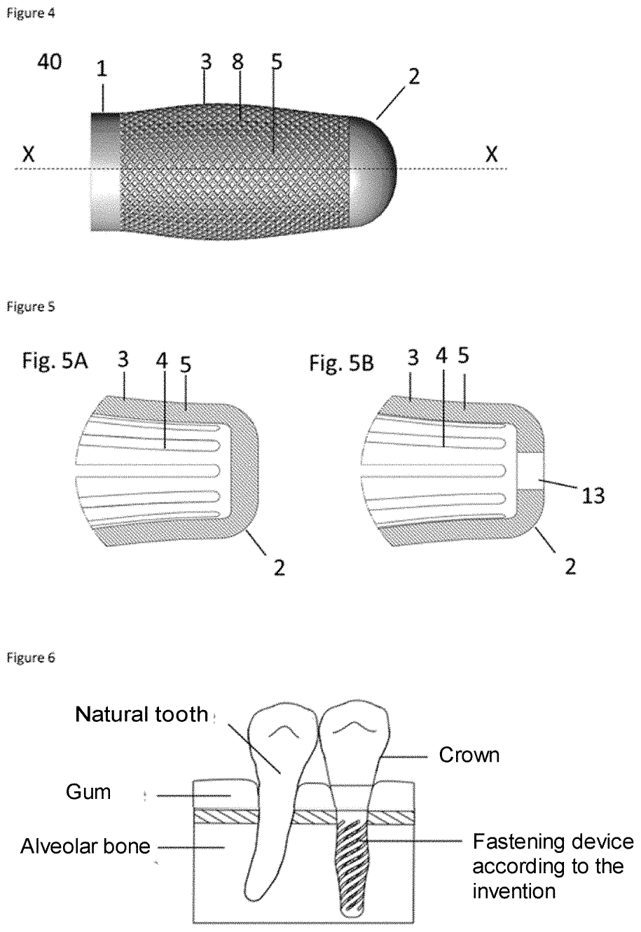

FIG. 4 shows a side view of the fastening device (40) for an implant, in particular a dental implant, according to another embodiment of the invention, in which the wall (5) of the hollow body (3) has a lattice (8).

FIGS. 5A-5B show views in partial lateral section of devices according to the invention, at the hollow body (3) and at the distal part (2). As depicted, the distal end (13) of the distal part (2) may be closed (FIG. 5A) or pierced (FIG. 5B).

FIGS. 6, 7 and 8 show particular ways of using the fastening device according to the invention, and in particular the implantation thereof in the jawbone, the knee joint or a finger joint.

FIG. 6 shows particularly a device according to the invention inserted into the alveolar bone of a jaw, and in which a dental implant is fastened, in order to form a dental prosthesis.

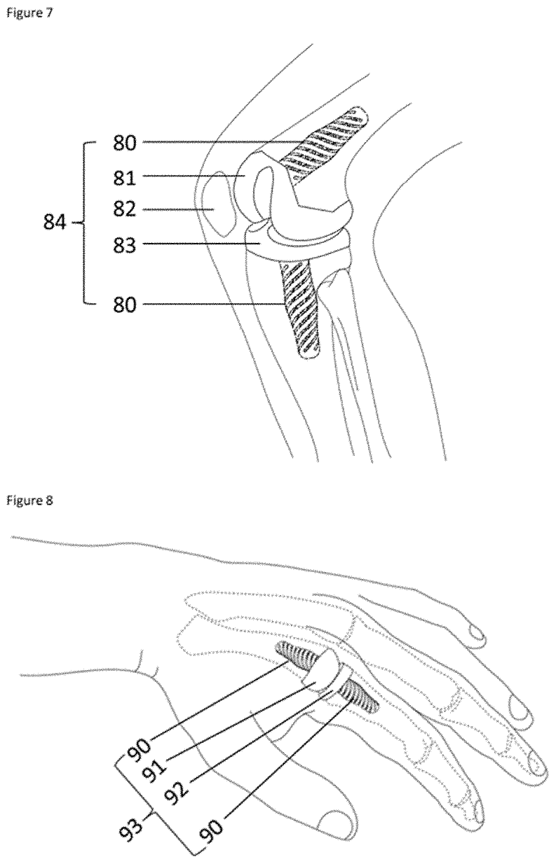

FIG. 7 shows a particular embodiment in which the fastening device (80) is inserted into a bone supporting the knee joint. In this context, the fastening device according to the invention is particularly designed to receive an implant intended to replace the knee joint. Thus, the articular prosthesis (84) is made up of two fastening devices according to the invention and of two complementary implants, one taking the form of the distal part of the femur (81) and the other the form of the proximal part of the tibia (82). The prosthesis may also comprise an artificial patella (83). According to one particular embodiment, the implant intended to be inserted into the fastening devices is in one piece and comprises an artificial knee joint made up of the elements (81), (82) and optionally (83).

FIG. 8 shows a particular embodiment in which the fastening device is inserted into a bone of a phalanx. In this context, the fastening device according to the invention is particularly designed to receive an implant intended to replace the metacarpophalangeal joint. Thus, the articular prosthesis (93) is made up of two fastening devices according to the invention and of two complementary implants, one taking the form of the distal part of a metacarpus (91) and the other taking the form of the proximal part of the first phalanx (92). According to one particular embodiment, the implant intended to be inserted into the fastening devices is in one piece and comprises an artificial metacarpophalangeal joint made up of the elements (91) and (92).

It should be noted that the present fastening device could be used for any other joints than the knee and the finger in order to restore a compromised function of the body.

1-22. (canceled)

23. A fastening device (10, 30, 40, 80, 90) for an implant, comprising an open proximal part (1), a distal part (2) and a central hollow body (3) extending between the proximal part (1) and the distal part (2), said hollow body (3) forming a housing (4) extending along a longitudinal axis X, said device being made of elastic material and said hollow body (3) being elastically deformable by axial elongation along the longitudinal axis X so as to pass from an unstressed state into a stressed state and vice versa, wherein a length L of the hollow body (3) in the unstressed state is less than a length L′ of said hollow body (3) in the stressed state, and a diameter D of the hollow body (3) in the unstressed state is greater than a diameter D′ of said hollow body (3) in the stressed state.

24. The device according to claim 23, wherein the hollow body (3) has a tubular overall shape.

25. The device according to claim 23, wherein a wall (5) of the hollow body delimiting the housing (4) is perforated.

26. The device according to claim 23, wherein the wall (5) of the hollow body (3) comprises at least one longitudinal slot (6) and/or at least one helical slot (7).

27. The device according to claim 26, wherein the wall (5) of the hollow body (3) comprises at least two helical slots with different or opposite pitches.

28. The device according to claim 23, wherein the wall of the hollow body is in the form of a lattice (8).

29. The device according to claim 23, wherein the distal part (2) has a conical or frustoconical cylindrical shape.

30. The device according to claim 23, wherein the distal end (12) of the distal part (2) is closed or pierced.

31. The device according to claim 23, wherein the wall (5) of the hollow body comprises a fastening surface intended for fastening an implant in said hollow body (3).

32. The device according to claim 23, wherein the elastic material is a polymer, titanium or one of its alloys, an alloy of niobium or an alloy of zirconium.

33. The device according to claim 23, wherein the elastic material is titanium or one of its alloys, and wherein a variation in length between the length L and the length L′ is between 0.1 and 2 millimetres and/or a variation in diameter between the diameter D and the diameter D′ is between 0.01 and 1 millimetre.

34. The device according to claim 23, wherein the elastic material is titanium or one of its alloys, and wherein the wall of the hollow body has a yield strength in tension of between 10⁸ and 10¹⁰ Pascal, and/or a Young's modulus of between 4×10¹⁰ and 30×10¹⁰ Pascal or between 6.5×10¹⁰ and 7×10¹⁰ Pascal.

35. The device according to claim 23, wherein the housing (4) of the hollow body (3) is able to receive a dental implant or an orthopaedic implant.

36. A kit comprising at least one fastening device (10, 30, 40, 80, 90) according to claim 23, an elastic deformation means configured to elastically deform the hollow body (3) of the fastening device (10, 30, 40, 80, 90) by axial elongation such that said hollow body (3) passes from a stressed state to an unstressed state or vice versa, and optionally at least one implant.

37. The kit according to claim 36, comprising:
a fastening device and a dental implant, or
two fastening devices and at least one implant, said implant being an articular implant.

38. The kit according to claim 37, comprising two fastening devices and two complementary implants, which are able to form an articular joint.

39. The kit according to claim 38, wherein:
a) a first implant is in the form of a distal part of a metacarpus and a second implant is in the form of a proximal part of a first phalanx, so as to reproduce a metacarpophalangeal joint; or
b) a first implant is in the form of a distal part of a femur and a second implant is in the form of a proximal part of a tibia, so as to reproduce a knee joint, the kit optionally further comprising an artificial patella.

40. A method for implanting a fastening device in a bone of a patient requiring an implant, comprising:
applying stress to a fastening device according to claim 23 (10) by axial stretching;
the insertion of the device (10) in a stressed state into an implant housing formed in a bone of the patient;
the releasing of said device (10) in the implant housing so as to lock the fastening device in close contact with a wall of the implant housing.

41. The method according to claim 40, said method comprising a preliminary step in which the implant housing is formed in a bone of the patient by drilling and/or boring.

42. The method according to claim 40, also comprising, after the fitting of the fastening device in the implant housing, a step of fastening an implant in said fastening device.
